(19) United States
(12) Patent Application Publication
(10) Pub. No.: US 2010/0167376 A1
(43) Pub. Date: Jul. 1, 2010

(54) METHODS FOR PROVIDING CELLULAR LYSATES FROM CELL WALL-CONTAINING SAMPLES

(75) Inventors: Michael Hogan, Tucson, AZ (US); Michael Saghbini, Poway, CA (US); Joseph Utermohlen, Tucson, AZ (US); Chunnian Shi, San Diego, CA (US)

Correspondence Address: COOLEY LLP;ATTN: Patent Group, Suite 1100, 777 - 6th Street, NW, WASHINGTON, DC, 20001, US

(73) Assignee: GenVault Corporation, Carlsbad, CA (US)

(21) Appl. No.: 12/574,676

(22) Filed: Oct. 6, 2009

Related U.S. Application Data
(60) Provisional application No. 61/103,222, filed on Oct. 6, 2008.

(51) Int. Cl. C12N 9/42; C12S 3/00; C12S 3/20; C12N 9/00

(52) U.S. Cl. 435/209; 435/267; 435/270; 435/183

(57) ABSTRACT

The present invention provides methods useful for making lysates from cell wall-containing cellular samples, including plant tissue samples and cultures of yeast or bacteria. The invention further provides compositions (e.g., solutions) that can be used in the methods of the invention, and kits comprising solutions and/or other reagents useful for carrying out the methods of the invention.

The present invention claims priority from U.S. Provisional Application No. 61/103,222, filed on Oct. 6, 2008, the contents of which are expressly incorporated herein by reference.

### BACKGROUND OF THE INVENTION

Nucleic acid extraction from plant tissues is complicated by the presence of a rigid cell wall that must be disrupted prior to recovery of cellular biomolecules (e.g., DNA, RNA, proteins, lipids and metabolites), and the presence of extremely durable biopolymers such as lignin, hemicellulose and cellulose, which comprise the structural lattice for the tissues. Traditionally, mechanical methods such as grinding in the presence of liquid nitrogen, grinding with metal or ceramic beads, and homogenization via high speed rotary blade cutting and sonication have been utilized for plant cell wall disruption and for shearing of the lignin and cellulosic plant tissue lattice. These traditional methods are labor intensive and require dedicated instrumentation that render the recovery of plant biomolecules expensive and difficult to automate for high throughput applications (e.g., in the area of applied genomics and proteomics). Recently, methods have been developed to reduce the amount of labor involved in extracting DNA from plant tissue, but such methods still require constant agitation and elevated temperatures.

There is a need in the field to develop additional methods for isolating nucleic acids and other biomolecules from plant tissues.

### SUMMARY OF THE INVENTION

The present invention is based, in part, on the discovery that certain enzymatic and pre-enzymatic solutions provide for quantitative recovery of biomolecules from cell wall-containing cellular samples in the absence of mechanical processing and elevated temperatures. The present invention is also based, in part, on the discovery that certain stabilization solutions preserve cell wall-containing cellular samples, allowing for quantitative recovery of biomolecules from such samples when the sample is not newly harvested and has not been frozen or freeze-dried. Accordingly, the present invention provides compositions, methods, and kits useful for stabilizing and making lysates from cell-wall containing cellular samples. The present invention also provides mixtures containing compositions of the invention and plant tissue or other cell-wall containing cellular samples.

In one aspect, the invention provides methods useful for making lysates from cell wall-containing cellular samples. In certain embodiments, the methods comprise incubating a cell wall-containing cellular sample in an enzymatic solution comprising at least one cell wall and tissue lattice degrading enzyme activity and a metal chelator. In certain embodiments, the enzymatic solution further comprises a preservative, a surfactant, or a combination thereof. In certain embodiments, the enzymatic solution further comprises a preservative, a surfactant, and either a buffer, a polyol (e.g., a short-chain polyol), or a combination thereof. In certain embodiments, the enzymatic solution has a pH of between about 4.0 and about 9.0.

In certain embodiments, the enzymatic incubation is carried out at ambient temperature (e.g., between 10° C. and 40° C.). In certain embodiments, the enzymatic incubation is carried out in the absence of mechanical processing. In certain embodiments, the enzymatic incubation is carried out at ambient temperature and in the absence of mechanical processing.

In certain embodiments, the cell wall-containing cellular sample is incubated in a pre-enzymatic solution prior to the enzymatic incubation. In certain embodiments, the cell wall-containing cellular sample is incubated in a stabilization solution prior to the enzymatic incubation. In certain embodiments, the cell wall-containing cellular sample is incubated in a stabilization solution followed by a pre-enzymatic solution prior to the enzymatic incubation.

In other embodiments, the methods comprise incubating a cell wall-containing cellular sample in an enzymatic solution comprising at least one cell wall and tissue lattice degrading enzyme activity and a metallic salt. In certain embodiments, the enzymatic solution further comprises at least one polyol, such as a short-chain polyol. In certain embodiments, the enzymatic solution further comprises a preservative, a surfactant, or a combination thereof. In certain embodiments, the enzymatic solution further comprises at least one polyol, and either a preservative, a surfactant, or a combination thereof. In certain embodiments, the enzymatic solution has a pH of between about 4.0 and about 9.0.

In certain embodiments, the enzymatic incubation is carried out at ambient temperature (e.g., between 10° C. and 40° C.). In certain embodiments, the enzymatic incubation is carried out in the absence of mechanical processing. In certain embodiments, the enzymatic incubation is carried out at ambient temperature and in the absence of mechanical processing.

In certain embodiments, lysates produced by the methods of the invention contain biomolecules, such as nucleic acids (e.g., DNA and/or RNA), proteins, carbohydrates, lipids, and cellular metabolites. In certain embodiments, the cell wall-containing cellular samples are samples obtained from a multicellular organism, such as a plant (e.g., a monocot or dicot), or consist of a population of single-cell organisms, such as bacteria or yeast.

In another aspect, the invention provides compositions (e.g., solutions) used in the methods of the invention. In certain embodiments, the composition is an enzymatic solution comprising at least one cell wall and tissue lattice degrading enzyme activity and a metal chelator. In certain embodiments, the enzymatic solution further comprises a preservative, a surfactant, or a combination thereof. In certain embodiments, the enzymatic solution further comprises a preservative, a surfactant, and either a buffer, a polyol (e.g., a short-chain polyol), or a combination thereof. In certain embodiments, the enzymatic solution has a pH of between about 4.0 and about 9.0.

In other embodiments, the composition is an enzymatic solution comprising at least one cell wall and tissue lattice degrading enzyme activity, at least one polyol, and a metallic salt. In certain embodiments, the enzymatic solution further comprises a preservative, a surfactant, or a combination thereof. In certain embodiments, the enzymatic solution has a pH of between about 4.0 and about 9.0.

In another aspect, the invention provides kits useful for carrying out the methods of the invention. In certain embodiments, the kits comprise a solution which, upon adding a cell wall and tissue lattice degrading enzyme preparation, becomes an enzymatic solution of the invention. In certain embodiments, the kits further comprise a cell wall and tissue lattice degrading enzyme preparation. In certain embodiments, the kits further comprise a stabilization solution, a pre-enzymatic solution, or a combination thereof. In certain embodiments, the kits further comprise a container, such as a tube or a multi-well plate, useful for performing the methods of the invention. In certain embodiments, the kits further comprise instructions for using the contents of the kits to prepare a lysate from a cell wall-containing cellular sample.

In another aspect, the invention provides mixtures. In certain embodiments, the mixtures comprise a cell-wall containing cellular sample, such as plant tissue or bacterial or yeast cells, mixed with an enzymatic solution of the invention. In certain embodiments, the mixtures comprise a cell-wall containing cellular sample, such as plant tissue or bacterial or yeast cells, mixed with a metal chelator, a preservative, a surfactant, and at least one cell wall and tissue lattice degrading enzyme activity. In certain embodiments, the mixtures comprise a cell-wall containing cellular sample, such as plant tissue or bacterial or yeast cells, mixed with a metal chelator, a preservative, a surfactant, a buffer and/or a polyol, and at least one cell wall and tissue lattice degrading enzyme activity.

In other embodiments, the mixtures comprise a cell-wall containing cellular sample, such as plant tissue or bacterial or yeast cells, mixed with a metallic salt, at least one polyol, and at least one cell-wall and tissue lattice degrading enzyme activity. In still other embodiments, the mixtures comprise a cell-wall containing cellular sample, such as plant tissue or bacterial or yeast cells, a metallic salt, at least one polyol, a preservative and/or a surfactant, and at least one cell-wall and tissue lattice degrading enzyme activity.

The invention and additional embodiments thereof will be set forth in greater detail in the detailed description that follows.

### BRIEF DESCRIPTION OF THE DRAWINGS

FIG. 1 shows DNA isolated from corn leaf disks, run out on a 0.8% agarose gel, and stained with ethidium bromide. The DNA was isolated as set forth in Example 2. In FIG. 1A, the lysate that the DNA was isolated from was prepared using an enzymatic solution comprising cellulase, hemicellulase, and pectinase activities. In FIG. 1B, the lysate that the DNA was isolated from as prepared using an enzymatic solution comprising cellulase and pectinase activities.

FIG. 2 shows DNA isolated from corn leaf tears, run out on a 0.8% agarose gel, and stained with ethidium bromide. The DNA was isolated as set forth in Example 3. In FIG. 2A, the lysate that the DNA was isolated from was prepared using an enzymatic solution comprising Tris, MES, calcium chloride, mannitol, sorbitol, boric acid, EDTA, and sarkosyl. In FIG. 2B, the lysate that the DNA was isolated from was prepared using enzymatic solution comprising Tris, mannitol, boric acid, EDTA, and sarkosyl. In FIG. 2C, the lysate that the DNA was isolated from was prepared using an enzymatic solution comprising Tris, boric acid, EDTA, and sarkosyl.

FIG. 3 shows DNA isolated from corn leaf tears, run out on a denaturing polyacrylamide gel, and stained with SYBR® gold. The DNA was isolated as set forth in Example 5.

### DETAILED DESCRIPTION OF THE INVENTION

As used herein, the following terms shall have the following meanings.

The term “biomolecules” is expressly intended to include short and long biopolymers including, but not limited to, such polymeric molecules as DNA, RNA, proteins, and carbohydrates, whether naturally existing or synthesized and with or without modified molecules, such as modified amino acids or nucleotides. Thus, for example, the term includes both short, oligomeric nucleic acid molecules (e.g., less than 50 bases in length), long nucleic acid molecules (e.g., greater than 50 kB in length), and any length in between. The term similarly encompasses both short peptide sequences (e.g., less than 10 amino acids), long polypeptide sequences (e.g., greater than 1000 amino acids in length), and any length in between. In addition, the term “biomolecules” is expressly intended to include small molecules found in biological samples, such as lipids, coenzymes, and metabolites.

The term “protein” as used herein is used interchangeably with the term “polypeptide.”

The term “nucleic acid,” “oligonucleotide” and “polynucleotide” are used interchangeably and encompass DNA, RNA, cDNA, whether single stranded or double stranded, as well as chemical modifications thereof.

Methods

The present invention is based, in part, on the discovery that certain enzymatic and pre-enzymatic solutions provide for quantitative recovery of biomolecules from cell wall-containing cellular samples in the absence of mechanical processing and elevated temperatures. The present invention is also based, in part, on the discovery that certain stabilization solutions preserve cell wall-containing cellular samples, allowing for quantitative recovery of biomolecules from such samples when the sample is not newly harvested and has not been frozen or freeze-dried.

Accordingly, in one aspect, the invention provides methods of making lysates from cell wall-containing cellular samples. As used herein, a “lysate” is a solution derived from the disruption of cells (e.g., cell wall-containing cells) and the release and/or solubilization of biomolecules contained within such cells. The biomolecules of a lysate can be found on or within the cytoplasm, nucleus, organelles, or membranes of the cells that are going to be lysed.

As used herein, a “cell wall-containing cellular sample” is a sample either from a multicellular organism, wherein the multicellular organism comprises cells that have cell walls, or from a population of cells, wherein the individual cells in the population have cell walls. In certain embodiments, the cell wall-containing cellular sample is a plant tissue sample. Examples of plant tissues include, but are not limited to, leaves (e.g., cotyledons, bracts, etc.), blades (e.g., from a grass), roots, seeds, seed cases, flowers, floral organs (e.g., sepals, petals, stamen, carpels), and fragments thereof. In certain embodiments, the plant tissue sample is from a seedling. In other embodiments, the plant tissue sample is from an adult plant.

In certain embodiments, the plant tissue sample is a leaf punch (e.g., a 6 mm leaf punch) or equivalent amount of cellular matter (e.g., roots, seed case shavings, floral organs, etc.). In certain embodiments, the plant tissue sample is from a leaf and has an area of about 10 mm² to about 100 mm², about 11 mm² to about 90 mm², about 12 mm² to about 80 mm², about 13 mm² to about 70 mm², about 14 mm² to about 60 mm², about 15 mm² to about 50 mm², about 16 mm² to about 40 mm², about 17 mm² to about 30 mm², or about 18 mm² to about 25 mm², or about 20 mm². In other embodiments, the plant tissue sample is from a leaf and has an area of about 10 mm² to about 500 mm², about 20 mm² to about 450 mm², about 30 mm² to about 400 mm², about 40 mm² to about 350 mm², about 50 mm² to about 300 mm², about 75 mm² to about 250 mm², about 100 mm² to about 200 mm², or about 125 mm² to about 175 mm², or about 140 mm² to about 160 mm².

In other embodiments, the plant tissue sample is an amount of cellular matter equivalent to a leaf area of about 10 mm² to about 100 mm², about 11 mm² to about 90 mm², about 12 mm² to about 80 mm², about 13 mm² to about 70 mm², about 14 mm² to about 60 mm², about 15 mm² to about 50 mm², about 16 mm² to about 40 mm², about 17 mm² to about 30 mm², or about 18 mm² to about 25 mm², or about 20 mm². In still other embodiments, the plant tissue sample is an amount of cellular matter equivalent to a leaf area of about 10 mm² to about 500 mm², about 20 mm² to about 450 mm², about 30 mm² to about 400 mm², about 40 mm² to about 350 mm², about 50 mm² to about 300 mm², about 75 mm² to about 250 mm², about 100 mm² to about 200 mm², or about 125 mm² to about 175 mm², or about 140 mm² to about 160 mm².

In certain embodiments, the plant tissue sample is intact. As used herein, the term “intact” refers to the plant tissue sample being free of mechanical processing (e.g., grinding, blending, and/or sonication). In certain embodiments, the plant tissue sample is completely intact (i.e., the sample has not been mechanically processed other than by being cut in order to be collected). For example, a leaf punch or a clipping from a leaf that is not otherwise cut or processed is completely intact. In contrast, a plant tissue sample that is intact, but not completely intact, can be cut or sliced beyond what is need to harvest the sample, so long as the general morphology of the sample is still evident.

In certain embodiments, the plant tissue sample is from a conifer, an angiosperm, a fern, or a lycophyte. Examples of angiosperm include, e.g., monocots, such as grasses (e.g., rice, wheat, maize, bamboo), palms, bananas, gingers, onions, or ornamentals (e.g., lilies, daffodils, irises, amaryllis, orchids, cannas, bluebells, tulips, etc.), and dicots, such as alfalfas, red clovers, green cresses, broccolis, cauliflowers, sunflowers, Diakon radishes, kales, mustards (including Arabidopsis thaliana), soys, canolas, camelias, tomatos, tobaccos, lentils, cabbages, and ornamentals (e.g., roses).

In other embodiments, the cell wall-containing cellular sample is a population of yeast cells, such as brewers yeast (e.g., S. cerevisiae, S. pombe, etc.) or infectious yeast (e.g., Candida, Blastomyces, etc.). In still other embodiments, the cell wall-containing sample is a population of bacterial cells, such as gram-negative bacteria (e.g., E. coli, Salmonella, Enterobacteriaceae, Pseudomonas, Moraxella, Helicobacter, Stenotrophomonas, Bdellovibrio, Legionella, Neisseria, Hemophilus, Klebsiella, Proteus, Enterobacter, Serratia, Acinetobacter, etc.), gram-positive bacteria (e.g., Bacillus, Listeria, Staphylococcus, Streptococcus, Enterococcus, Clostridium, etc.), or mycobacterium (e.g., M. tuberculosis).

In certain embodiments, the population of cells (e.g., yeast, bacterial, or mycobacterial cells) comprises an amount of cellular matter equivalent in mass to a leaf area of about 10 mm² to about 100 mm², about 11 mm² to about 90 mm², about 12 mm² to about 80 mm², about 13 mm² to about 70 mm², about 14 mm² to about 60 mm², about 15 mm² to about 50 mm², about 16 mm² to about 40 mm², about 17 mm² to about 30 mm², or about 18 mm² to about 25 mm², or about 20 mm². In still other embodiments, the population of cells (e.g., yeast, bacterial, or mycobacterial cells) comprises an amount of cellular matter equivalent in mass to a leaf area of about 10 mm² to about 500 mm², about 20 mm² to about 450 mm², about 30 mm² to about 400 mm², about 40 mm² to about 350 mm², about 50 mm² to about 300 mm², about 75 mm² to about 250 mm², about 100 mm² to about 200 mm², or about 125 mm² to about 175 mm², or about 140 mm² to about 160 mm².

In certain embodiments, the cell wall-containing cellular sample is fresh. As used herein, a “fresh” sample is a sample that has been harvested within two hours of an enzymatic or pre-enzymatic incubation. In certain embodiments, a fresh sample has been harvested within 90, 75, 60, 45, 30, 25, 20, 15, 10, 5, 4, 3, 2 minutes, or less, prior to an enzymatic or pre-enzymatic incubation. In other embodiments, the cell wall-containing cellular sample is freeze-dried or frozen prior to an incubation. In still other embodiments, the cell wall-containing cellular sample has been stabilized (i.e., incubated in a stabilization solution described herein) prior to being incubated with an enzymatic or pre-enzymatic solution.

In certain embodiments, the methods comprise incubating a cell wall-containing cellular sample in an enzymatic solution (i.e., performing an enzymatic incubation), wherein said enzymatic solution comprises at least one cell wall and tissue lattice degrading enzyme activity. As used herein, a “cell wall and tissue lattice degrading enzyme activity” is an enzymatic activity arising from one or more enzymes capable of breaking down at least one component of a cell wall, such as cellulose, hemicellulose, pectin, or lignin.

In certain embodiments, the cell wall and tissue lattice degrading enzyme activity comprises a cellulase activity. The cellulase activity can arise from one or more enzymes capable of breaking down cellulose, whether or not the enzyme is formally identified as a cellulase. Enzymes capable of breaking down cellulose include, but are not limited to, endocellulases, exocellulases, cellobiases, and oxidative cellulases. Endocellulases go by various names, including endo-1,4-beta-glucanase, carboxymethyl cellulase (CMCase), endo-1,4-beta-D-glucanase, Beta-1,4-glucanase, Beta-1,4-endoglucan hydrolase, and celludextrinase; exocellulases are also called cellobiohydrolases; and cellobiases are also called beta-glucosidases. Accordingly, in certain embodiments, the cell wall and tissue lattice degrading enzyme activity comprises a cellulase activity, wherein said cellulase activity comprises at least one (e.g., two or more) endocellulase enzyme, at least one (e.g., two or more) exocellulase enzyme, at least one (e.g., two or more) cellobiase enzyme, or at least one (e.g., two or more) oxidative cellulase enzyme. The cell wall and tissue lattice degrading enzyme activity can also include a cellulase activity arising from a mixture of two or more different types of enzymes that are capable of breaking down cellulose. For example, in certain embodiments, the cellulase activity comprises at least one endocellulase enzyme and at least one exocellulase enzyme. In other embodiments, the cellulase activity comprises at least one endocellulase enzyme, at least one exocellulase enzyme, and at least one cellobiase enzyme. In other embodiments, the cellulase activity comprises at least one endocellulase enzyme, at least one exocellulase enzyme, and at least one oxidative cellulase enzyme. In still other embodiments, the cellulase activity comprises at least one endocellulase enzyme, at least one exocellulase enzyme, at least one cellobiase enzyme, and at least one oxidative cellulase enzyme.

In other embodiments, the cell wall and tissue lattice degrading enzyme activity comprises a pectinase activity. The pectinase activity can arise from one or more enzymes capable of breaking down pectin, whether or not the enzyme is formally identified as a pectinase. Enzymes capable of breaking down pectin include, but are not limited to, pectinlyases, pectinesterases, polygalacturonases, and arabinases. Accordingly, in certain embodiments, the cell wall and tissue lattice degrading enzyme activity comprises a pectinase activity, wherein said pectinase activity comprises at least one (e.g., two or more) pectinlyase enzyme, at least one (e.g., two or more) pectinesterase enzyme, at least one (e.g., two or more) polygalacturonase enzyme, or at least one (e.g., two or more) arabinase enzyme. The cell wall and tissue lattice degrading enzyme activity can also include a pectinase activity arising from a mixture of two or more different types of enzymes that are capable of breaking down pectin. For example, in certain embodiments, the pectinase activity comprises at least one pectinlyase enzyme and at least one polygalacturonase enzyme. In other embodiments, the pectinase activity comprises at least one pectinlyase enzyme and at least one arabinase enzyme. In other embodiments, the pectinase activity comprises at least one arabinase enzyme and at least one polygalacturonase enzyme. In other embodiments, the pectinase activity comprises at least one pectinlyase enzyme, at least one polygalacturonase enzyme, and at least one pectinesterase enzyme. In other embodiments, the pectinase activity comprises at least one pectinlyase enzyme, at least one arabinase enzyme, and at least one pectinesterase enzyme. In other embodiments, the pectinase activity comprises at least one arabinase enzyme, at least one polygalacturonase enzyme, and at least one pectinesterase enzyme. In still other embodiments, the pectinase activity comprises at least one pectinlyase enzyme, at least one polygalacturonase enzyme, at least one arabinase enzyme, and at least one pectinesterase enzyme.

In other embodiments, the cell wall and tissue lattice degrading enzyme activity comprises a hemicellulase activity. The hemicellulase activity can arise from one or more enzymes capable of breaking down hemicellulose, whether or not the enzyme is formally identified as a hemicellulase. Enzymes capable of breaking down hemicellulose include, but are not limited to, endoarabinases, endoarabinogalactanases, endoglucanases, endomannanases, endoxylanases, feraxan endoxylanases, feruloyl esterases, xylosidases, exomannosidases, glucuronidases, galactosidases, endogalactanases, and acetyl xylanesterases. Accordingly, in certain embodiments, the cell wall and tissue lattice degrading enzyme activity comprises a hemicellulase activity, wherein said hemicellulase activity comprises at least one (e.g., two or more) endoarabinase enzyme, at least one (e.g., two or more) endoarabinogalactanase enzyme, at least one (e.g., two or more) endoglucanase enzyme, at least one (e.g., two or more) endomannanase enzyme, at least one (e.g., two or more) endoxylanase enzyme, at least one (e.g., two or more) feraxan endoxylanase enzyme, at least one (e.g., two or more) feruloyl esterase enzyme, at least one (e.g., two or more) xylosidase enzyme, at least one (e.g., two or more) exomannosidase enzyme, at least one (e.g., two or more) glucuronidase enzyme, at least one (e.g., two or more) galactosidase enzyme, at least one (e.g., two or more) endogalactanase enzyme, or at least one (e.g., two or more) acetyl xylanesterase enzyme. The cell wall and tissue lattice degrading enzyme activity can also include a hemicellulase activity arising from a mixture of two or more different types of enzymes that are capable of breaking down hemicellulose. For example, in certain embodiments, the hemicellulase activity comprises two or more enzymes selected from the group consisting of endoarabinases, endoarabinogalactanases, endoglucanases, endomannanases, endoxylanases, feraxan endoxylanases, feruloyl esterases, xylosidases, exomannosidases, glucuronidases, galactosidases, endogalactanases, and acetyl xylanesterases.

In still other embodiments, the cell wall and tissue lattice degrading enzyme activity comprises a ligninase activity. The ligninase activity can arise from one or more enzymes capable of breaking down lignin, whether or not the enzyme is formally identified as a ligninase. Enzymes capable of breaking down lignin include, but are not limited to, phenol oxidases (e.g., laccase-type phenol oxidases) and peroxidases (e.g., lignin peroxidases, manganese peroxidases, versatile peroxidases). Accordingly, in certain embodiments, the cell wall and tissue lattice degrading enzyme activity comprises a ligninase activity, wherein said ligninase activity comprises at least one (e.g., two or more) phenol oxidase enzyme, or at least one (e.g., two or more) peroxidase enzyme. The cell wall and tissue lattice degrading enzyme activity can also include a ligninase activity arising from a mixture of different types of enzymes that are capable of breaking down lignin. For example, in certain embodiments, the ligninase activity comprises at least one phenol oxidase enzyme and at least one peroxidase enzyme.

In certain embodiments, the cell wall and tissue lattice degrading enzyme activity comprises a cellulase activity and a pectinase activity. The cellulase activity can arise from one or more enzymes capable of breaking down cellulose, as discussed above. The pectinase activity can arise from one or more enzymes capable of breaking down pectin, as discussed above. Accordingly, in certain embodiments, the cell wall and tissue lattice degrading enzyme activity comprises a mixture of one or more enzymes capable of breaking down cellulose (e.g., one or more endocellulases, one or more exocellulases, one or more cellobiases, one or more oxidative cellulases, or any combination thereof, such as one or more endocellulases and one or more exocellulases) and one or more enzymes capable of breaking down pectin (e.g., one or more pectinlyases, one or more pectinesterases, one or more polygalacturonases, one or more arabinases, or any combination thereof, such as one or more pectinlyases and one or more polygalacturonases).

In certain embodiments, the cell wall and tissue lattice degrading enzyme activity comprises a cellulase activity, a hemicellulase activity, and a pectinase activity. The cellulase activity can arise from one or more enzymes capable of breaking down cellulose, as discussed above. The hemicellulase activity can arise from one or more enzymes capable of breaking down hemicellulose, as discussed above. The pectinase activity can arise from one or more enzymes capable of breaking down pectin, as discussed above. Accordingly, in certain embodiments, the cell wall and tissue lattice degrading enzyme activity comprises a mixture of one or more enzymes capable of breaking down cellulose (e.g., one or more endocellulases, one or more exocellulases, one or more cellobiases, one or more oxidative cellulases, or any combination thereof, such as one or more endocellulases and one or more exocellulases), one or more enzymes capable of breaking down hemicellulose (e.g., one or more endoarabinases, one or more endoarabinogalactanases, one or more endoglucanases, one or more endomannanases, one or more endoxylanases, one or more feraxan endoxylanases, one or more feruloyl esterases, one or more xylosidases, one or more exomannosidases, one or more glucuronidases, one or more galactosidases, one or more endogalactanases, one or more acetyl xylanesterases, or any combination thereof), and one or more enzymes capable of breaking down pectin (e.g., one or more pectinlyases, one or more pectinesterases, one or more polygalacturonases, one or more arabinases, or any combination thereof, such as one or more pectinlyases and one or more polygalacturonases).

In certain embodiments, the cell wall and tissue lattice degrading enzyme activity comprises a cellulase activity, a hemicellulase activity, a pectinase activity, and a ligninase activity. The cellulase activity can arise from one or more enzymes capable of breaking down cellulose, as discussed above. The hemicellulase activity can arise from one or more enzymes capable of breaking down hemicellulose, as discussed above. The pectinase activity can arise from one or more enzymes capable of breaking down pectin, as discussed above. The ligninase activity can arise from one or more enzymes capable of breaking down lignin, as discussed above. Accordingly, in certain embodiments, the cell wall and tissue lattice degrading enzyme activity comprises a mixture of one or more enzymes capable of breaking down cellulose (e.g., one or more endocellulases, one or more exocellulases, one or more cellobiases, one or more oxidative cellulases, or any combination thereof, such as one or more endocellulases and one or more exocellulases), one or more enzymes capable of breaking down hemicellulose (e.g., one or more endoarabinases, one or more endoarabinogalactanases, one or more endoglucanases, one or more endomannanases, one or more endoxylanases, one or more feraxan endoxylanases, one or more feruloyl esterases, one or more xylosidases, one or more exomannosidases, one or more glucuronidases, one or more galactosidases, one or more endogalactanases, one or more acetyl xylanesterases, or any combination thereof), one or more enzymes capable of breaking down pectin (e.g., one or more pectinlyases, one or more pectinesterases, one or more polygalacturonases, one or more arabinases, or any combination thereof, such as one or more pectinlyases and one or more polygalacturonases), and one or more enzymes capable of breaking down lignin (e.g., one or more phenol oxidases, one or more peroxidases, or any combination thereof).

In certain embodiments, the cell wall and tissue lattice degrading enzyme activity comprises two or more enzymes selected form the group consisting of cellulases, xylanases, beta-glucanases, pectinases, glycosidases, mannases, xyloglucanases, ferulic acid esterases, and combinations thereof. In certain embodiments, the cell wall and tissue lattice degrading enzyme activity comprises at least one cellulase enzyme (e.g., one or more endocellulases, one or more exocellulases, or any combination thereof), at least one pectinase enzyme (e.g., one or more pectinlyases, one or more pectinesterases, one or more polygalacturonases, one or more arabinases, or any combination thereof), and at least one enzyme selected form the group consisting of xylanases, beta-glucanases, glycosidases, mannases, xyloglucanases, ferulic acid esterases, and combinations thereof.

In certain embodiments, the cell wall and tissue lattice degrading enzyme activity comprises a mixture of enzymes from an organism that naturally produces cell wall degrading enzymes (e.g., a microbe or fungus that consumes and/or decomposes plant matter). In certain embodiments, the organism has been selected and/or genetically engineered to produce a more active cell wall and tissue lattice degrading enzyme activity. In certain embodiments, the cell wall and tissue lattice degrading enzyme activity comprises a mixture of enzymes from two or more different organisms (e.g., two or more microbial or fungal organisms that naturally produce cell wall degrading enzymes). Suitable organisms include, but are not limited to, Aspergillus (e.g., Aspergillus niger, Aspergillus sp.), Penicillium (e.g., Penicillium frequentans, Penicillium expansum, Penicillium griseoroseum), Trichoderma (e.g., Tricoderma viride, Tricoderma harzianum), Humicola (e.g., Humicola insolence), and Rhizoctonia (e.g., Rhizoctonia solan).

Commercially available enzyme preparations that can be used in the methods and enzymatic solutions of the invention include, for example, lysing enzymes from Asergillus sp., lysing enzymes from Trichoderma harzianum, lysing enzymes from Rhizoctonia solan (sold by Sigma®), and Viscozyme® L. Commercially available cellulase preparation that can be used in the methods and enzymatic solutions of the invention include, but are not limited to, Aspergillus niger cellulase, Aspergillus sp. cellulase, Humicola insolence cellulase, Trichoderma viride cellulase, CloneZyme™ cellulase, CelluSeb-TL™, Cellulase 13L, Depol™ 692, and Celluclast™. Commercially available pectinase preparations that can be used in the methods and enzymatic solutions of the invention include Aspergillus niger pectinase (sold by Sigma®), Macer8™ FJ, pectinase 62L, pectinase 444L, Pectinex® Ultra SP-L, and Depol™ 20L.

In certain embodiments, the enzymatic solution comprises an Aspergillus niger cellulase preparation and a pectinase preparation selected from the group consisting of Aspergillus tiger pectinase, Macer8™ FJ, pectinase 62L, pectinase 444L, Pectinex® Ultra SP-L, and Depol™ 20L. In other embodiments, the enzymatic solution comprises an Aspergillus sp. cellulase preparation and a pectinase preparation selected from the group consisting of Aspergillus niger pectinase, Macer8™ FJ, pectinase 62L, pectinase 444L, Pectinex® Ultra SP-L, and Depol™ 20L. In other embodiments, the enzymatic solution comprises a Humicola insolence cellulase preparation and a pectinase preparation selected from the group consisting of Aspergillus niger pectinase, Macer8™ FJ, pectinase 62L, pectinase 444L, Pectinex® Ultra SP-L, and Depol™ 20L. In other embodiments, the enzymatic solution comprises a Trichoderma viride cellulase preparation and a pectinase preparation selected from the group consisting of Aspergillus niger pectinase, Macer8™ FJ, pectinase 62L, pectinase 444L, Pectinex® Ultra SP-L, and Depol™ 20L. In other embodiments, the enzymatic solution comprises a CloneZyme™ cellulase preparation and a pectinase preparation selected from the group consisting of Aspergillus niger pectinase, Macer8™ FJ, pectinase 62L, pectinase 444L, Pectinex® Ultra SP-L, and Depol™ 20L. In other embodiments, the enzymatic solution comprises a CelluSeb-TL™ preparation and a pectinase preparation selected from the group consisting of Aspergillus niger pectinase, Macer8™ FJ, pectinase 62L, pectinase 444L, Pectinex® Ultra SP-L, and Depol™ 20L. In other embodiments, the enzymatic solution comprises a Cellulase 13L preparation and a pectinase preparation selected from the group consisting of Aspergillus niger pectinase, Macer8™ FJ, pectinase 62L, pectinase 444L, Pectinex® Ultra SP-L, and Depol™ 20L. In other embodiments, the enzymatic solution comprises a Depol™ 692 preparation and a pectinase preparation selected from the group consisting of Aspergillus niger pectinase, Macer8™ FJ, pectinase 62L, pectinase 444L, Pectinex® Ultra SP-L, and Depol™ 20L. In still other embodiments, the enzymatic solution comprises a Celluclast™ preparation and a pectinase preparation selected from the group consisting of Aspergillus niger pectinase, Macer8™ FJ, pectinase 62L, pectinase 444L, Pectinex® Ultra SP-L, and Depol™ 20L.

In certain embodiments, the enzymatic solution comprises about 1% to about 50%, about 2% to about 40%, about 3% to about 30%, or about 5% to about 25% of an cell wall and tissue lattice degrading enzyme preparation (e.g., a commercially available enzyme preparation or mixture of commercially available enzyme preparations). For example, in certain embodiments, the enzymatic solution comprises about 2% to about 30%, about 3% to about 25%, about 4% to about 20%, or about 5% to about 15% of a cellulase preparation (e.g., Aspergillus niger cellulase, Aspergillus sp. cellulase, Humicola insolence cellulase, Trichoderma virile cellulase, CloneZyme™ cellulase, CelluSeb-TL™, Cellulase 13L, Depol™ 692, or Celluclast™). In other embodiments, the enzymatic solution comprises about 0.5% to about 20%, about 0.75% to about 15%, about 1.0% to about 10%, about 1.25% to about 7.5%, or about 1.5% to about 5.0% of a pectinase preparation (e.g., Aspergillus niger pectinase, Macer8™ FJ, pectinase 62L, pectinase 444L, Pectinex® Ultra SP-L, or Depol™ 20L). In other embodiments, the enzymatic solution comprises about 2% to about 30%, about 3% to about 25%, about 4% to about 20%, or about 5% to about 15% of a cellulase preparation in combination with about 0.5% to about 20%, about 0.75% to about 15%, about 1.0% to about 10%, about 1.25% to about 7.5%, or about 1.5% to about 5.0% of a pectinase preparation. In certain embodiments, the cellulase preparation has an activity of about 500 U/g to about 2000 U/g, about 600 U/g to about 1500 U/g, or about 700 U/g to about 1000 U/g. In certain embodiments, the pectinase preparation has an activity (e.g., endogalacturonase activity) of about 500 U/g to about 2000 U/g, about 1000 U/g to about 1750 U/g, or about 1500 U/g.

In certain embodiments, the enzymatic solution comprises a metal chelator. Examples of suitable metal chelators include, but are not limited to, EDTA, EGTA, o-phenanthroline, and a crown ether (e.g., a tetramer (e.g., 12-crown-4), pentamer (e.g., 15-crown-5), or hexamer (e.g., 18-crown-6) crown ether), an aza-crown equivalent, or a mixed amine-ether crown equivalent. In certain embodiments, the concentration of metal chelator in the enzymatic solution is about 5 mM to about 150 mM, about 10 mM to about 125 mM, about 15 mM to about 100 mM, about 20 mM to about 75 mM, about 25 mM to about 60 mM, about 30 mM to about 50 mM, about 35 mM to about 45 mM, or about 40 mM. In other embodiments, the concentration of metal chelator in the enzymatic solution is about 5 mM to about 150 mM, about 10 mM to about 125 mM, about 20 mM to about 100 mM, about 30 mM to about 90 mM, about 40 mM to about 80 mM, about 50 mM to about 75 mM, about 60 mM to about 70 mM, about 65 mM to about 69 mM, or about 67 mM. In certain embodiments, the concentration of metal chelator in the enzymatic solution is about 5 mM to about 150 mM, about 10 mM to about 140 mM, about 20 mM to about 130 mM, about 40 mM to about 120 mM, about 60 mM to about 115 mM, about 80 mM to about 110 mM, about 90 mM to about 105 mM, or about 100 mM. In still other embodiments, the concentration of metal chelator in the enzymatic solution is about 50 mM to about 200 mM, about 75 mM to about 175 mM, about 90 mM to about 160 mM, about 100 mM to about 150 mM, about 110 mM to about 140 mM, about 120 mM to about 130 mM, or about 125 mM.

In certain embodiments, the enzymatic solution comprises a preservative (e.g., an acidic preservative). In certain embodiments, the preservative is selected from the group consisting of borate (or borax), phosphate, vanadate, alum (e.g., potassium alum (KAl(SO₄)₂.12H₂O), soda alum (Na₂SO₄Al₂(SO₄)₃.24H₂O), or ammonium alum (NH₄Al(SO₄)₂.12H₂O)), or an acid thereof (e.g., boric acid, phosphoric acid, etc.). In certain embodiments, the preservative is present in a concentration of about 1 mM to about 500 mM, about 2 mM to about 400 mM, about 3 mM to about 300 mM, about 4 mM to about 200 mM, about 5 mM to about 100 mM, about 6 mM to about 75 mM, about 7 mM to about 60 mM, about 8 mM to about 50 mM, about 9 mM to about 40 mM, about 10 mM to about 30 mM, about 15 mM to about 25 mM, or about 20 mM. In other embodiments, the preservative is present in a concentration of about 1 mM to about 500 mM, about 2 mM to about 400 mM, about 3 mM to about 300 mM, about 4 mM to about 250 mM, about 5 mM to about 200 mM, about 6 mM to about 175 mM, about 7 mM to about 150 mM, about 8 mM to about 125 mM, about 9 mM to about 100 mM, about 10 mM to about 90 mM, about 15 mM to about 80 mM, about 20 mM to about 70 mM, about 25 mM to about 60 mM, about 30 mM to about 50 mM, or about 40 mM. In still other embodiments, the preservative is present in a concentration of about 1 mM to about 500 mM, about 2 mM to about 400 mM, about 3 mM to about 300 mM, about 4 mM to about 250 mM, about 5 mM to about 200 mM, about 10 mM to about 180 mM, about 15 mM to about 160 mM, about 20 mM to about 140 mM, about 30 mM to about 130 mM, about 40 mM to about 120 mM, about 50 mM to about 110 mM, about 60 mM to about 100 mM, about 70 mM to about 90 mM, about 75 mM to about 85 mM, or about 80 mM. In certain embodiments, the preservative is borate or boric acid.

In certain embodiments, the enzymatic solution further comprises a surfactant (e.g., a non-ionic detergent or an ionic detergent). In certain embodiments, the surfactant is a non-ionic detergent comprising polyoxypropylene and/or polyoxyethylene, such as Triton X-100, NP-40, Igepal CA-630, Tween 20, Tween 80, a Brij detergent, n-dodecyl-b-D-maltoside, etc. In certain embodiments, the surfactant is an ionic detergent such as sarcosyl. In certain embodiments, the surfactant (e.g., Triton X-100 or sarcosyl) is present in the enzymatic solution at a concentration between about 0.1% to about 20%, about 0.2% to about 10%, about 0.25% to about 5%, about 0.3% to about 3.0%, about 0.35% to about 1.5%, about 0.4% to about 1%, or about 0.5%. In other embodiments, the surfactant (e.g., Triton X-100 or sarcosyl) is present in the enzymatic solution at a concentration between about 0.1% to about 20%, about 0.2% to about 10%, about 0.3% to about 5%, about 0.4% to about 2.5%, about 0.5% to about 1.5%, or about 1%. In still other embodiments, the surfactant (e.g., Triton X-100 or sarcosyl) is present in the enzymatic solution at a concentration between about 0.1% to about 20%, about 0.25% to about 10%, about 0.5% to about 5%, about 0.75% to about 3.0%, about 1.0% to about 2.0%, or about 1.5%.

In certain embodiments, the enzymatic solution has a pH between about 4.0 and about 9.0, about 4.1 and about 7.0, about 4.2 and about 6.0, about 4.3 and about 5.5, about 4.4 and about 5.0, or about 4.5. In other embodiments, the enzymatic solution has a pH between about 4.0 and about 9.0, about 4.1 and about 8.5, about 4.2 and about 8.0, about 4.3 and about 7.5, about 4.4 and about 7.0, about 4.5 and about 6.5, about 4.6 and about 6.0, about 4.7 and about 5.5, about 4.8 and about 5.25, or about 5.0. In still other embodiments, the enzymatic solution has a pH between about 4.0 and about 9.0, about 4.5 and about 8.5, about 5.0 and about 8.0, about 5.5 and about 7.5, about 5.75 and about 7.25, between about 6.0 and about 7.0, or about 6.1.

In certain embodiments, the enzymatic solution comprises a buffer (e.g., a buffer that maintains the pH of the enzymatic solution within the desired range). In certain embodiments, the enzymatic solution comprises a buffer selected from the group consisting of 2-(cyclohexylamino) ethanesulfonic acid (CHES), N-(2-hydroxyethyl)piperazine-N′-(3-propanesulfonic acid) (EPPS), N-(2-hydroxyethyl)piperazine-N-(2-ethanesulfonic acid (HEPES), 2-(N-morpholino) ethanesulfonic acid (MES), 3-(N-morpholino) propanesulfonic acid (MOPS), piperazine-N,N′-bis(2-ethanesulfonic acid (PIPES), [(2-hydroxy-1,1-bis[bydroxymethyl]ethyl)amino]-1-propanesulfonic acid (TAPS), ethanolamine, 3-amino-1-propanesulfonic acid, and 2-amino-2-hydroxymethyl-1,3-propanediol (Tris). In certain embodiments, the enzymatic solution comprises a buffer having a concentration of about 1 mM to about 250 mM, about 2 mM to about 200 mM, about 3 mM to about 150 mM, about 4 mM to about 100 mM, about 5 mM to about 50 mM, about 6 mM to about 40 mM, about 7 mM to about 30 mM, about 8 mM to about 20 mM, about 9 mM to about 15 mM, or about 10 mM. In other embodiments, the enzymatic solution comprises a buffer having a concentration of about 1 mM, about 2 mM, about 3 mM, about 4 mM, about 5 mM, about 6 mM, about 7 mM, about 8 mM, about 9 mM, or about 10 mM.

In certain embodiments, the enzymatic solution comprises a MES buffer having a concentration of about 1 mM or about 100 mM, about 2 mM to about 80 mM, about 3 mM to about 60 mM, about 4 mM to about 40 mM, or about 5 mM to about 20 mM. In other embodiments, the enzymatic solution comprises a MES buffer having a concentration of about 1 mM, about 2 mM, about 3 mM, about 4 mM, about 5 mM, about 6 mM, about 7 mM, or about 8 mM. In certain embodiments, the enzymatic solution comprises a Tris buffer having a concentration of about 1 mM or about 100 mM, about 2 mM to about 80 mM, about 3 mM to about 60 mM, about 4 mM to about 40 mM, or about 5 mM to about 20 mM. In other embodiments, the enzymatic solution comprises a Tris buffer having a concentration of about 1 mM, about 2 mM, about 3 mM, about 4 mM, about 5 mM, about 6 mM, about 7 mM, or about 8 mM. In certain embodiments, the enzymatic solution comprises a combination of buffers, such as MES and Tris (e.g., about 1 mM to about 10 mM, about 2 mM to about 8 mM, or about 3 mM, about 4 mM, about 5 mM, about 6 mM, about 7 mM, or about 8 mM MES, in combination with about 1 mM to about 20 mM, about 2 mM to about 15 mM, about 3 mM to about 10 mM, or about 3 mM, about 4 mM, about 5 mM, about 6 mM, about 7 mM, about 8 mM, about 9 mM, or about 10 mM Tris).

In certain embodiments, the enzymatic solution further comprises a metallic salt. In certain embodiments, the metallic salt comprises calcium (e.g., CaCl₂, calcium phosphate). In other embodiments, the metallic salt comprises magnesium (e.g., MgCl₂). In certain embodiments, the metallic salt is present at a concentration of about 0.5 mM to about 20 mM, about 0.75 mM to about 15 mM, about 1.0 mM to about 10 mM, about 1.25 mM to about 5 mM, about 1.5 mM to about 2.5 mM, or about 2 mM. In other embodiments, the metallic salt is present at a concentration of about 1 mM to about 20 mM, about 2 mM to about 15 mM, about 2.5 mM to about 10 mM, about 3 mM to about 5 mM, or about 4 mM. In still other embodiments, the metallic salt is present at a concentration of about 1 mM to about 20 mM, about 2 mM to about 15 mM, about 3 mM to about 12.5 mM, about 4 mM to about 10 mM, about 6 mM to about 9 mM, or about 8 mM.

In certain embodiments, the enzymatic solution further comprises at least one polyol. In certain embodiments, the polyol is a short-chain polyol. In certain embodiments, the short-chain polyol is selected from the group consisting of ethylene glycol, 1-3 propane diol, glycerol, butane triol (e.g., n-butane triol or isobutane triol), erythritol, pentane triol (e.g., n-pentane triol or isopentane triol), pentane tetraol (e.g., n-pentane tetraol, isopentane tetraol), pentaerythritol, xylitol, sorbitol, and mannitol. In certain embodiments, the enzymatic solution further comprises at least two short-chain polyols (e.g., mannitol and another short-chain polyol described herein, such as sorbitol). In other embodiments, the polyol is a monosaccharide, a disaccharide, or a polymer (e.g., dextran) having a molecular weight less than 10 kDa. In certain embodiments, the polyol is present at a concentration of about 0.2% to about 10%, about 0.4% to about 7.5%, about 0.5% to about 5.0%, about 0.6% to about 2.5%, about 0.7% to about 1.0%, or about 0.75%. In other embodiments, the polyol is present at a concentration of about 0.5% to about 10%, about 1.0% to about 7.5%, about 1.2% to about 5.0%, about 1.4% to about 2.5%, or about 1.6%. In other embodiments, the polyol is present at a concentration of about 0.5% to about 10%, about 1.0% to about 7.5%, about 1.5% to about 5.0%, about 2.0% to about 4.0%, about 2.5% to about 3.5%, or about 3.0%. In still other embodiments, the polyol is present at a concentration of about 1% to about 10%, about 2% to about 7.5%, about 2.5% to about 5.0%, or about 3.0% to about 4.0%. In certain embodiments, the enzymatic solution comprises mannitol, sorbitol, or a combination thereof. In other embodiments, the enzymatic solution comprises glycerol.

In certain embodiments, the enzymatic solution comprises at least one cell wall and tissue lattice degrading enzyme activity and a metal chelator (e.g., at a concentration of about 5 mM to about 150 mM). In other embodiments, the enzymatic solution comprises at least one cell wall and tissue lattice degrading enzyme activity, a metal chelator (e.g., at a concentration of about 5 mM to about 150 mM), a preservative (e.g., at a concentration of about 5 mM to about 200 mM), and a surfactant (e.g., at a concentration of about 0.2% to about 10%). In other embodiments, the enzymatic solution comprises at least one cell wall and tissue lattice degrading enzyme activity, a metal chelator at a concentration of about 5 mM to about 150 mM), a preservative (e.g., at a concentration of about 5 mM to about 200 mM), a surfactant (e.g., at a concentration of about 0.2% to about 10%), and a buffer (e.g., at a concentration of about 1 mM to about 100 mM). In other embodiments, the enzymatic solution comprises at least one cell wall and tissue lattice degrading enzyme activity, a metal chelator (e.g., at a concentration of about 5 mM to about 150 mM), a preservative (e.g., at a concentration of about 5 mM to about 200 mM), a surfactant (e.g., at a concentration of about 0.2% to about 10%), and a polyol (e.g., a short-chain polyol at a concentration of about 0.5% to about 10%). In still other embodiments, the enzymatic solution comprises at least one cell wall and tissue lattice degrading enzyme activity, a metal chelator (e.g., at a concentration of about 5 mM to about 150 mM), a preservative (e.g., at a concentration of about 5 mM to about 200 mM), a surfactant (e.g., at a concentration of about 0.2% to about 10%), a buffer (e.g., at a concentration of about 1 mM to about 100 mM), and a polyol (e.g., a short-chain polyol at a concentration of about 0.5% to about 10%). In related embodiments, each of the enzymatic solutions of this paragraph comprises at least two cell wall and tissue lattice degrading enzyme activities (e.g., a cellulase activity, a pectinase activity, and, optionally, a hemicellulase activity or a ligninase activity). In other related embodiments, each of the enzymatic solutions of this paragraph comprises at least three cell wall and tissue lattice degrading enzyme activities (e.g., a cellulase activity, a pectinase activity, a hemicellulase activity, and, optionally, a ligninase activity).

In certain embodiments, the enzymatic solution comprises 6× mM MES, 7.5× mM Tris, 6× mM CaCl₂, 60× mM B(OH)₃, 2.4X % sorbitol, 2.4X % mannitol, 1.5X % sarcosyl, 6Y % (v/v) of a cellulase preparation (e.g., Aspergillus niger cellulase, Aspergillus sp. cellulase, Humicola insolence cellulase, Trichoderma viride cellulase, CloneZyme™ cellulase, CelluSeb-TL™, Cellulase 13L, Depol™ 692, or Celluclast™), and 1.5Y % (v/v) of a pectinase preparation (e.g., Aspergillus niger pectinase, Macer8™ FJ, pectinase 62L, pectinase 444L, Pectinex® Ultra SP-L, or Depol™ 20L), wherein said enzymatic solution has a pH of about 4.0 to about 6.5, wherein X has a value between about 0.5 and about 1.5, and wherein Y has a value between about 1 and about 7. In certain embodiments, X has a value selected from 0.5, 0.67, 1.0, 1.33, and 1.5, and Y has a value selected from 0.23, 0.34, 0.67, 1, 1.34, 1.67, 2.67, 4, 5.3, and 6.67. In certain embodiments, the enzymatic solutions of this paragraph further comprise 100× mM of a metal chelator (e.g., EDTA or EGTA).

In certain embodiments, the enzymatic solution comprises 6× mM MES, 7.5× mM Tris, 6× mM CaCl₂, 60× mM B(OH)₃, 5X % glycerol, 1.5X % sarcosyl, 6Y % (v/v) of a cellulase preparation (e.g., Aspergillus niger cellulase, Aspergillus sp. cellulase, Humicola insolence cellulase, Trichoderma viride cellulase, CloneZyme™ cellulase, CelluSeb-TL™, Cellulase 13L, Depol™ 692, or Celluclast™) and 1.5Y % (v/v) of a pectinase preparation (e.g., Aspergillus niger pectinase, Macer8™ FJ, pectinase 62L, pectinase 444L, Pectinex® Ultra SP-L, or Depol™ 20L), wherein said enzymatic solution has a pH of about 4.0 to about 6.5, wherein X has a value between about 0.5 and about 1.5, and wherein Y has a value between about 1 and about 7. In certain embodiments, X has a value selected from 0.5, 0.67, 1.0, 1.33, and 1.5, and Y has a value selected from 0.23, 0.34, 0.67, 1, 1.34, 1.67, 2.67, 4, 5.3, and 6.67. In certain embodiments, the enzymatic solutions of this paragraph further comprise 100× mM of a metal chelator (e.g., EDTA or EGTA).

In certain embodiments, the enzymatic solution comprises 6× mM MES, 7.5× mM Tris, 6× mM CaCl₂, 60× mM B(OH)₃, 1.5X % sarcosyl, 6Y % (v/v) of a cellulase preparation (e.g., Aspergillus niger cellulase, Aspergillus sp. cellulase, Humicola insolence cellulase, Trichoderma viride cellulase, CloneZyme™ cellulase, CelluSeb-TL™, Cellulase 13L, Depol™ 692, or Celluclast™), and 1.5Y % (v/v) of a pectinase preparation (e.g., Aspergillus niger pectinase, Macer8™ FJ, pectinase 62L, pectinase 444L, Pectinex® Ultra SP-L, or Depol™ 20L), wherein said enzymatic solution has a pH of about 4.0 to about 6.5, wherein X has a value between about 0.5 and about 1.5, and wherein Y has a value between about 1 and about 7. In certain embodiments, X has a value selected from 0.5, 0.67, 1.0, 1.33, and 1.5, and Y has a value selected from 0.23, 0.34, 0.67, 1, 1.34, 1.67, 2.67, 4, 5.3, and 6.67. In certain embodiments, the enzymatic solutions of this paragraph further comprise 100× mM of a metal chelator (e.g., EDTA or EGTA).

In certain embodiments, the enzymatic solution comprises 6× mM MES, 7.5× mM Tris, 60× mM B(OH)₃, 1.5X % sarcosyl, 6Y % (v/v) of a cellulase preparation (e.g., Aspergillus niger cellulase, Aspergillus sp. cellulase, Humicola insolence cellulase, Trichoderma viride cellulase, CloneZyme™ cellulase, CelluSeb-TL™, Cellulase 13L, Depol™ 692, or Celluclast™), and 1.5Y % (v/v) of a pectinase preparation (e.g., Aspergillus niger pectinase, Macer8™ FJ, pectinase 62L, pectinase 444L, Pectinex® Ultra SP-L, or Depol™ 20L), wherein said enzymatic solution has a pH of about 4.0 to about 6.5, wherein X has a value between about 0.5 and about 1.5, and wherein Y has a value between about 1 and about 7. In certain embodiments, X has a value selected from 0.5, 0.67, 1.0, 1.33, and 1.5, and Y has a value selected from 0.23, 0.34, 0.67, 1, 1.34, 1.67, 2.67, 4, 5.3, and 6.67. In certain embodiments, the foregoing enzymatic solutions further comprise 100× mM of a metal chelator (e.g., EDTA or EGTA).

In certain embodiments, the enzymatic solution comprises 7.50× mM Tris, 60× mM B(OH)₃, 1.5X sarcosyl, 6Y % (v/v) of a cellulase preparation (e.g., Aspergillus niger cellulase, Aspergillus sp. cellulase, Humicola insolence cellulase, Trichoderma viride cellulase, CloneZyme™ cellulase, CelluSeb-TL™, Cellulase 13L, Depol™ 692, or Celluclast™), and 1.5Y % (v/v) of a pectinase preparation (e.g., Aspergillus niger pectinase, Macer8™ FJ, pectinase 62L, pectinase 444L, Pectinex® Ultra SP-L, or Depol™ 20L), wherein said enzymatic solution has a pH of about 4.0 to about 6.5, wherein X has a value between about 0.5 and about 1.5, and wherein Y has a value between about 1 and about 7. In certain embodiments, X has a value selected from 0.5, 0.67, 1.0, 1.33, and 1.5, and Y has a value selected from 0.23, 0.34, 0.67, 1, 1.34, 1.67, 2.67, 4, 5.3, and 6.67. In certain embodiments, the foregoing enzymatic solutions further comprise 100× mM of a metal chelator (e.g., EDTA or EGTA).

In certain embodiments, the enzymatic solution comprises 60× mM B(OH)₃, 1.5X % sarcosyl, 6Y % (v/v) of a cellulase preparation (e.g., Aspergillus niger cellulase, Aspergillus sp. cellulase, Humicola insolence cellulase, Trichoderma viride cellulase, CloneZyme™ cellulase, CelluSeb-TL™, Cellulase 13L, Depol™ 692, or Celluclast™), and 1.5Y % (v/v) of a pectinase preparation (e.g., Aspergillus niger pectinase, Macer8™ FJ, pectinase 62L, pectinase 444L, Pectinex® Ultra SP-L, or Depol™ 20L), wherein said enzymatic solution has a pH of about 4.0 to about 6.5, wherein X has a value between about 0.5 and about 1.5, and wherein Y has a value between about 1 and about 7. In certain embodiments, X has a value selected from 0.5, 0.67, 1.0, 1.33, and 1.5, and Y has a value selected from 0.23, 0.34, 0.67, 1, 1.34, 1.67, 2.67, 4, 5.3, and 6.67. In certain embodiments, the foregoing enzymatic solutions further comprise 100× mM of a metal chelator (e.g., EDTA or EGTA).

In certain embodiments, the enzymatic solution comprises at least one cell wall and tissue lattice degrading enzyme activity and a metal chelator (e.g., at a concentration of about 50 mM to about 200 mM). In other embodiments, the enzymatic solution comprises at least one cell wall and tissue lattice degrading enzyme activity, a metal chelator (e.g., at a concentration of about 50 mM to about 200 mM), and a buffer (e.g., at a concentration of about 50 mM to about 200 mM)). In other embodiments, the enzymatic solution comprises at least one cell wall and tissue lattice degrading enzyme activity, a metal chelator (e.g., at a concentration of about 50 mM to about 200 mM), a buffer (e.g., at a concentration of about 50 mM to about 200 mM), and an acid (e.g., at a concentration of about 25 mM to about 100 mM). In related embodiments, each of the enzymatic solutions of this paragraph comprises at least two cell wall and tissue lattice degrading enzyme activities (e.g., a cellulase activity, a pectinase activity, and, optionally, a hemicellulase activity or a ligninase activity). In other related embodiments, each of the enzymatic solutions of this paragraph comprises at least three cell wall and tissue lattice degrading enzyme activities (e.g., a cellulase activity, a pectinase activity, a hemicellulase activity, and, optionally, a ligninase activity).

In certain embodiments, the enzymatic solution comprises 100× mM MES, 100× mM EDTA, 40× mM citric acid, 6Y % (v/v) of a cellulase preparation (e.g., Aspergillus niger cellulase, Aspergillus sp. cellulase, Humicola insolence cellulase, Trichoderma viride cellulase, CloneZyme™ cellulase, CelluSeb-TL™, Cellulase 13L, Depol™ 692, or Celluclast™), and 1.5Y % (v/v) of a pectinase preparation (e.g., Aspergillus niger pectinase, Macer8™ FJ, pectinase 62L, pectinase 444L, Pectinex® Ultra SP-L, or Depol™ 20L), wherein said enzymatic solution has a pH of about 4.0 to about 6.5, wherein X has a value between about 0.5 and about 1.5, and wherein Y has a value between about 1 and about 7. In certain embodiments, X has a value selected from 0.5, 0.67, 1.0, 1.33, and 1.5, and Y has a value selected from 0.23, 0.34, 0.67, 1, 1.34, 1.67, 2.67, 4, 5.3, and 6.67.

In certain embodiments, the enzymatic solution comprises 25 mM MES, pH 6.5, 25 mM EDTA, and 10% (v/v) of a cellulase preparation (e.g., Aspergillus niger cellulase, Aspergillus sp. cellulase, Humicola insolence cellulase, Trichoderma viride cellulase, CloneZyme™ cellulase, CelluSeb-TL™, Cellulase 13L, Depol™ 692, or Celluclast™). In certain embodiments, the enzymatic solution comprises 25 mM Tris, pH 6.5, 25 mM EDTA, and 10% (v/v) of a cellulase preparation. In certain embodiments, the enzymatic solution comprises 25 mM MES, pH 6.5, 37.5 mM EDTA, and 10% (v/v) of a cellulase preparation. In certain embodiments, the enzymatic solution comprises 25 mM Tris, pH 6.5, 37.5 mM EDTA, and 10% (v/v) of a cellulase preparation.

In certain embodiments, the enzymatic solution comprises 25 mM MES, pH 6.5, 25 mM EDTA, 25% (v/v) of a cellulase preparation (e.g., Aspergillus niger cellulase, Aspergillus sp. cellulase, Humicola insolence cellulase, Trichoderma viride cellulase, CloneZyme™ cellulase, CelluSeb-TL™, Cellulase 13L, Depol™ 692, or Celluclast™), and 17.5% (v/v) pectinase preparation (e.g., Aspergillus niger pectinase, Macer8™ FJ, pectinase 62L, pectinase 444L, Pectinex® Ultra SP-L, and Depol™ 20L). In certain embodiments, the enzymatic solution comprises 25 mM Tris, pH 6.5, 25 mM EDTA, 25% (v/v) of a cellulase preparation, and 17.5% (v/v) of a pectinase preparation. In certain embodiments, the enzymatic solution comprises 25 mM Tris, pH 6.5, 37.5 mM EDTA, 25% (v/v) of a cellulase preparation, and 17.5% (v/v) of a pectinase preparation. In certain embodiments, the enzymatic solution comprises 25 mM Tris, pH 6.5, 37.5 mM EDTA, 25% (v/v) of a cellulase preparation, and 17.5% (v/v) of a pectinase preparation.

Typically, the cell wall-containing cellular sample is incubated in an amount of enzymatic solution sufficient to wet the sample and allow for enzymatic digestion to occur efficiently, without diluting the sample excessively. In certain embodiments, the cell wall-containing cellular sample is incubated with the enzymatic solution in a volume to volume ratio of about 1:2 to about 1:1000, about 1:5 to about 1:800, about 1:10 to about 1:700, about 1:20 to about 1:600, about 1:40 to about 1:500, about 1:50 to about 1:400, about 1:60 to about 1:300, about 1:70 to about 1:250, about 1:80 to about 1:200, about 1:85 to about 1:175, about 1:90 to about 1:150, about 1:95 to about 1:125, or about 1:100. In certain embodiments, the cell wall-containing cellular sample is incubated in a volume of enzymatic solution of about 100 ul to about 1000 ul, about 150 ul to about 750 ul, about 200 ul to about 600 ul, or about 200 ul, about 250 ul, about 300 ul, about 350 ul, about 400 ul, about 450 ul, about 500 ul, about 550 ul, or about 600 ul.

In certain embodiments, the cell wall-containing cellular sample is incubated in the enzymatic solution for at least 8, 10, 12, 13, 14, 15, 16, 17, 18, 19, 20, 21, 22, 23, 24, or more hours. In other embodiments, the cell wall-containing cellular sample is incubated in the enzymatic solution for about 1, 2, 3, 4, 5, 6, 7, or more days. In certain embodiments, the cell wall-containing cellular sample is incubated in the enzymatic solution while the cellular sample is being shipped (e.g., to a distant location for further processing).

In certain embodiments, the enzymatic incubation is performed at ambient temperature. As used herein, “ambient temperature” is the temperature in the location where the incubation is being performed (e.g., field, laboratory room, shipping vehicle, or otherwise). Typically, ambient temperature is between about 10° C. and about 40° C., about 15° C. and about 35° C., about 20° C. and about 30° C., or about 25° C. (e.g., room temperature). In other embodiments, the enzymatic incubation is performed at a temperature higher than ambient temperature, but less than, e.g., 50° C., 45° C., 40° C., 37° C., 35° C., 34° C., 33° C., 32° C., 31° C., 30° C., or 29° C.

In certain embodiments, the enzymatic incubation is performed in the absence of mechanical processing. As used herein, the phrase “mechanical processing” refers to substantial mechanical manipulation of the cell wall-containing cellular sample, such as grinding (e.g., with metal or ceramic beads), blending, and/or sonication. As used herein, “mechanical processing” does not include minor mechanical manipulations or agitation (e.g., pipetting or mixing) that occurs during the normal course of setting up and conducting an enzymatic incubation. In addition, “mechanical processing” does not include intermittent agitation, or even continuous, mild agitation during the course of an enzymatic incubation (e.g., sample agitation that occurs during sample movement and/or shipping).

In certain embodiments, the enzymatic incubation comprises intermittent agitation of the mixture of cell wall-containing cellular sample and enzymatic solution. For example, in certain embodiments, the mixture of cell wall-containing cellular sample and enzymatic solution is agitated (e.g., by vortexing, shaking, or flicking the vessel holding the mixture) at the end of the incubation (e.g., sometime during the last hour, half-hour, 25, 20, 15, 10, or 5 minutes of the incubation). In other embodiments, the mixture of cell wall-containing cellular sample and enzymatic solution is agitated (e.g., by vortexing, shaking, or flicking the vessel holding the mixture) intermittently during the incubation (e.g., at the beginning, middle, and/or end of the incubation). Such intermittent agitation can be continuous or pulsed, and can last for 10, 5, 4, 3, 2 minutes or less (e.g., less than a minute). In other embodiments, the mixture of cell wall-containing cellular sample and enzymatic solution is gently, but continuously, agitated (e.g., using a nutator, rolling drum, or other gentle shaking device) during the course of the incubation. In still other embodiments, the mixture of cell wall-containing cellular sample and enzymatic solution is agitated, but not continuously, during the course of the enzymatic incubation.

In certain embodiments, a flocculator is added to the enzymatic solution at the end of the enzymatic incubation (e.g., to clarify the lysate). In certain embodiments, the flocculator is a clay (e.g., a phyllosilicate clay, such as bentonite, montinorillonite, kaolinite, illite, pyrophyllite, etc.). In certain embodiments, the flocculator is added to the enzymatic solution to a concentration between about 3 mg/ml to about 80 mg/ml, about 4 mg/ml to about 60 mg/ml, about 5 mg/ml to about 40 mg/ml, about 6 mg/ml to about 20 mg/ml, about 7 mg/ml to about 10 mg/ml, or about 8 mg/ml. In certain embodiments, the flocculator is added to the enzymatic solution in an amount of about 0.1% to about 5.0%, about 0.2% to about 4.0%, about 0.3% to about 3.0%, about 0.4% to about 2.5%, about 0.5% to about 2.0%, about 0.6% to about 1.5%, about 0.7% to about 1.0%, or about 0.8% to about 0.9%. In certain embodiments, the flocculator and enzymatic solution mixture is agitated (e.g., vortexed, shaken, or otherwise mixed) at the end of the enzymatic incubation. In certain embodiments, the flocculator and enzymatic solution mixture is centrifuged (e.g., after the mixture is agitated) and the supernatant (i.e., clarified lysate) is then removed (e.g., decanted or pipetted away) so as to separate the pelleted flocculatent mass from the clarified lysate.

In certain embodiments, an extraction solution is added to the enzymatic solution at the end of the enzymatic incubation. In certain embodiments, the extraction solution comprises a non-ionic detergent comprising polyoxypropylene and/or polyoxyethylene, such as Triton X-100, NP-40, Igepal CA-630, Tween 20, Tween 80, Brij, n-dodecyl-b-D-maltoside, etc. In certain embodiments, the non-ionic detergent is present in the extraction solution at a concentration between about 0.1% to about 5%, about 0.2% to about 4%, about 0.3% to about 3%, about 0.4% to about 2%, about 0.5% to about 1.5%, or about 1%. In certain embodiments, the enzymatic incubation is allowed to proceed (e.g., at ambient temperature, or whatever temperature the enzymatic incubation was occurring at) following the addition of extraction solution. In certain embodiments, the enzymatic incubation proceeds for another 15, 30, 45, 60, 75, 90, 105, 120, or more minutes following addition of extraction solution.

In certain embodiments, the cell wall-containing cellular sample is incubated with a pre-enzymatic solution prior to the enzymatic incubation (i.e., a “pre-enzymatic incubation” is performed). In certain embodiments, the pre-enzymatic solution has a pH between about 10 and about 15, about 10.5 and about 14.9, about 11 and about 14.8, about 11.5 and about 14.7, about 12 and about 14.6, about 12.5 and about 14.5, about 13 and about 14.4, between about 13.5 and about 14.3. In certain embodiments, the pre-enzymatic incubation is carried out for about 0.5 hours to about 8 hours, about 1 hour to about 6 hours, about 1.5 hours to about 4 hours, or about 2 hours to about 3 hours.

In certain embodiments, the pre-enzymatic solution has a hydroxide ion concentration between about 0.02M to about 5M, about 0.1M to about 4M, about 0.5M to about 3.5M, about 1M to about 3M, about 1.5M to about 2.5M, or about 2M.

In certain embodiments, the pre-enzymatic solution comprises a base selected from the group consisting of a hydroxide salt, an amide salt, a carbanion salt, and a hydride salt. In certain embodiments, the pre-enzymatic solution comprises a base selected from the group consisting of KOH, Ba(OH)₂, CsOH, NaOH, Sr(OH)₂, Ca(OH)₂, LiOH, RbOH, NH₄OH, NaNH₂, NaH, and lithium diisopropyl amide (LiNC₆H₁₄).

In certain embodiments, the cell wall-containing cellular sample is incubated with the pre-enzymatic solution in a volume to volume ratio of about 1:10 to about 1:1000, about 1:20 to about 1:800, about 1:30 to about 1:700, about 1:40 to about 1:600, about 1:50 to about 1:500, about 1:60 to about 1:400, about 1:70 to about 1:300, about 1:75 to about 1:250, about 1:80 to about 1:200, about 1:85 to about 1:175, about 1:90 to about 1:150, about 1:95 to about 1:125, or about 1:100. In certain embodiments, the cell wall-containing cellular sample is incubated in a volume of pre-enzymatic solution of about 100 ul to about 1000 ul, about 150 ul to about 750 ul, about 200 ul to about 600 ul, or about 200 ul, about 250 ul, about 300 ul, about 350 ul, about 400 ul, about 450 ul, about 500 ul, about 550 ul, or about 600 ul.

In certain embodiments, the pre-enzymatic incubation is stopped prior to the beginning of the enzymatic incubation. In certain embodiments, stopping the pre-enzymatic incubation comprises neutralizing (e.g., partially or fully neutralizing) the pre-enzymatic solution. In certain embodiments, the pre-enzymatic solution is neutralized by the addition of an acidic solution (e.g., hydrochloric acid, citric acid, acetic acid, phosphoric acid, sulfuric acid, etc.). In certain embodiments, the pre-enzymatic solution is neutralized by the addition of an amount of an acidic solution that reduces the pH of the pre-enzymatic solution to a pH between about 5 and about 9, about 5.5 and about 8.0, about 5.75 to about 7.5, about 6.0 to about 7.0. In certain embodiments, the pre-enzymatic solution is neutralized by the addition of an acidic solution comprising hydrogen ions in an equimolar amount relative to the amount of hydroxide ions in the pre-enzymatic solution.

In certain embodiments, stopping the pre-enzymatic incubation comprises removing (e.g., decanting, aspirating, or pipetting away) the pre-enzymatic solution from the cell wall-containing cellular sample. In certain embodiments, stopping the pre-enzymatic incubation comprises neutralizing the pre-enzymatic solution and then removing the neutralized pre-enzymatic solution.

In certain embodiments, the cell wall-containing cellular sample is washed following removal of pre-enzymatic solution or neutralized pre-enzymatic solution. In certain embodiments, the wash solution consists of water. In other embodiments, the wash solution comprises a salt. In still other embodiments, the wash solution comprises a buffer, e.g., a buffer present in the enzymatic solution, such as a buffer selected from the group consisting of 2-(cyclohexylamino) ethanesulfonic acid (CHES), N-(2-hydroxyethyl)piperazine-N′-(3-propanesulfonic acid) (EPPS), N-(2-hydroxyethyl)piperazine-N′-(2-ethanesulfonic acid (HEPES), 2-(N-morpholino) ethanesulfonic acid (MES), 3-(N-morpholino) propanesulfonic acid (MOPS), piperazine-N,N′-bis(2-ethanesulfonic acid (PIPES), [(2-hydroxy-1,1-bis[hydroxymethyl]ethyl)amino]-1-propanesulfonic acid (TAPS), ethanolamine, 3-amino-1-propanesulfonic acid, and 2-amino-2-hydroxymethyl-1,3-propanediol (Tris).

In certain embodiments, the cell wall-containing cellular sample is incubated with a stabilization solution prior to the enzymatic and/or pre-enzymatic incubation. In certain embodiments, the stabilization solution comprises an alcohol, a short-chain polyol, or a combination thereof. In certain embodiments, the alcohol is a primary, secondary, or tertiary alcohol, such as methanol, ethanol, propanol (n-propanol or isopropanol), butanol (n-butanol or iosbutanol), etc. In certain embodiments, the short-chain polyol is soluble in alcohol (e.g., ethanol or propanol). In certain embodiments, the short-chain polyol is selected from the group consisting of ethylene glycol, 1-3 propane diol, glycerol, butane triol (e.g., n-butane triol or isobutane triol), erythritol, pentane triol (e.g., n-pentane triol or isopentane triol), pentane tetraol (e.g., n-pentane tetraol, isopentane tetraol), pentaerythritol, xylitol, and sorbitol.

In certain embodiments, the stabilization solution comprises about 50% to about 75% ethanol. In other embodiments, the stabilization solution comprises about 50% to about 75% isopropanol. In other embodiments, the stabilization solution comprises about 50% to about 75% ethanol and the remainder of the solution is a polyol that is soluble in ethanol (e.g., ethylene glycol or glycerol). For example, in certain embodiments, the stabilization solution comprises about 50% ethanol and about 50% ethylene glycol. In still other embodiments, the stabilization solution comprises about 50% to about 75% isopropanol and the remainder of the solution is a polyol that is soluble in isopropanol (e.g., ethylene glycol or glycerol).

In certain embodiments, the cell wall-containing cellular sample is incubated in stabilization solution for at least 8, 12, 16, 20, 24 hrs, or at least 2, 2.5, 3, 3.5, 4 days, or more. In certain embodiments, the cell wall-containing cellular sample is incubated with the stabilization solution in a volume to volume ratio of about 1:10 to about 1:1000, about 1:20 about 1:800, about 1:30 to about 1:700, about 1:40 to about 1:600, about 1:50 to about 1:500, about 1:60 to about 1:400, about 1:70 to about 1:300, about 1:75 to about 1:250, about 1:80 to about 1:200, about 1:85 to about 1:175, about 1:90 to about 1:150, about 1:95 to about 1:125, or about 1:100.

The lysates produced by the methods of the invention comprise, e.g., nucleic acids suitable for genetic analysis. Thus, the lysates can be cleaned up by standard methods (e.g., Qiagen columns or Whatman glass fiber filters), yielding high-quality nucleic acids that can be used for a variety of different assays, including PCR, quantitative PCR, SNP analysis, microarray analysis, nucleic acid sequencing, etc. Similarly, other biomolecules present in the lysate, including proteins, lipids, carbohydrates, and metabolites, can be isolated and analyzed using standard methodologies (e.g., ELISA, chromatography, thin layer chromatography, mass spectrometry, spectroscopy (e.g., absorption, fluorescence, etc.), spectrometry, etc.).

In certain embodiments, the methods of the invention provide for quantitative recovery of biomolecules from the cell wall-containing cellular samples. As used herein, the term “quantitative” refers to reproducible recovery of at least 90% or more of the biomolecules present in the original cell wall-containing cellular samples. In other embodiments, the methods of the invention provide for recovery of at least 70%, 75%, 80%, 85%, 90%, 95%, 98%, or more of one or more types of biomolecules (e.g., nucleic acid, protein, lipid, carbohydrate, or metabolite) present in the original cell wall-containing cellular samples.

Compositions

In another aspect, the invention provides compositions, wherein the compositions are solutions used in carrying out the methods of the invention (e.g., enzymatic solutions, pre-enzymatic solutions, etc.).

In certain embodiments, the composition is an enzymatic solution described herein. For example, in certain embodiments, the composition is an enzymatic solution comprising at least one cell wall and tissue lattice degrading enzyme activity and a metal chelator. In certain embodiments, the composition is an enzymatic solution comprising at least one cell wall and tissue lattice degrading enzyme activity, a metal chelator, a preservative, and a surfactant. In certain embodiments, the composition is an enzymatic solution comprising at least one cell wall and tissue lattice degrading enzyme activity, a metal chelator, a preservative, a surfactant, and a buffer. In certain embodiments, the composition is an enzymatic solution comprising at least one cell wall and tissue lattice degrading enzyme activity, a metal chelator, a preservative, a surfactant, and a polyol, such as a short-chain polyol. In certain embodiments, the composition is an enzymatic solution comprising at least one cell wall and tissue lattice degrading enzyme activity, a metal chelator, a preservative, a surfactant, a buffer, and a polyol, such as a short-chain polyol. In certain embodiments, the enzymatic solution comprises at least two cell wall and tissue lattice degrading enzyme activities (e.g., a cellulase activity and a pectinase activity). In certain embodiments, the enzymatic solution comprises at least three cell wall and tissue lattice degrading enzyme activities (e.g., a cellulase activity, a hemicellulase activity, a pectinase activity, and, optionally, a ligninase activity). In certain embodiments, the enzymatic solution has a pH of between about 4.0 and about 9.0, or about 4.1 to about 6.5.

In other embodiments, the composition is an enzymatic solution comprising at least one cell wall and tissue lattice degrading enzyme activity, at least one polyol, and a metallic salt. In certain embodiments, the composition is an enzymatic solution comprising at least one cell wall and tissue lattice degrading enzyme activity, at least one polyol, a metallic salt, and either a preservative, a surfactant, or a combination thereof. In certain embodiments, the enzymatic solution comprises at least two cell wall and tissue lattice degrading enzyme activities (e.g., a cellulase activity and a pectinase activity). In certain embodiments, the enzymatic solution comprises at least three cell wall and tissue lattice degrading enzyme activities (e.g., a cellulase activity, a hemicellulase activity, a pectinase activity, and, optionally, a ligninase activity). In certain embodiments, the enzymatic solution has a pH of between about 4.0 and about 9.0, or about 4.1 to about 6.5.

In certain embodiments, the composition is an enzymatic solution described herein, except that it lacks the cell wall and tissue lattice degrading enzyme activity.

Kits

In another aspect, the invention provides kits useful for carrying out the methods of the invention. In certain embodiments, the kits comprise an enzymatic solution of the invention. The solution can be any enzymatic solution described herein. In other embodiments, the kits comprise a solution which, upon adding a cell wall and tissue lattice degrading enzyme preparation, becomes an enzymatic solution of the invention. The solution can be any solution based on any enzymatic solution described herein.

In certain embodiments, the kits further comprise a cell wall and tissue lattice degrading enzyme preparation. The enzyme preparation can be any enzyme preparation described herein (e.g., a cellulase preparation, a pectinase preparation, a combination thereof, or a commercially available enzyme mixture). In certain embodiments, the kits further comprise a stabilization solution, a pre-enzymatic solution, or a combination thereof. The stabilization and pre-enzymatic solutions can be any stabilization and pre-enzymatic solutions described herein. In certain embodiments, the kits further comprise a detergent, for example, a nonionic detergent such as Triton X-100, or an ionic detergent such as sarcosyl. In certain embodiments, the kits further comprise a container, such as a tube or a multi-well plate, useful for performing the methods of the invention. In certain embodiments, the kits further comprise instructions for using the contents of the kits to prepare a lysate from a cell wall-containing cellular sample.

Mixtures

In another aspect, the invention provides mixtures. In certain embodiments, the mixtures comprise a cell-wall containing cellular sample, such as plant tissue or bacterial or yeast cells, and an enzymatic solution of the invention. The enzymatic solution can be any enzymatic solution described herein. For example, in certain embodiments, the mixtures comprise a cell-wall containing cellular sample, mixed with a metal chelator, a preservative, a surfactant, and at least one cell wall and tissue lattice degrading enzyme preparation. In other embodiments, the mixtures comprise a cell-wall containing cellular sample, such as plant tissue or bacterial or yeast cells, mixed with a metal chelator, a preservative, a surfactant, a buffer and/or a polyol, and a cell wall and tissue lattice degrading enzyme preparation. In other embodiments, the mixtures comprise a cell-wall containing cellular sample, such as plant tissue or bacterial or yeast cells, mixed with a metallic salt, at least one polyol, and a cell-wall and tissue lattice degrading enzyme preparation. In still other embodiments, the mixtures comprise a cell-wall containing cellular sample, such as plant tissue or bacterial or yeast cells, mixed with a metallic salt, at least one polyol, a preservative and/or a surfactant, and a cell-wall and tissue lattice degrading enzyme preparation.

The following examples are intended to illustrate, but not to limit, the invention in any manner, shape, or form, either explicitly or implicitly. While they are typical of those that might be used, other procedures, methodologies, or techniques known to those skilled in the art may alternatively be used.

### EXAMPLES

### Example 1

### Direct Liquefaction Protocol

One method of the invention for providing a cellular lysate from a cell wall-containing cellular sample is as follows:
1. Add 600 microliters of an enzymatic solution of the invention to a leaf tear that has been placed in a microfuge tube or well in a 96-well plate.
2. Incubate two days at ambient temperature (e.g., room temperature in a lab or ambient temperature during transport), without agitation.
3. Following the incubation, gently tap the tube to disintegrate the cell wall-containing cellular sample.
4. Add flocculator (e.g., bentonite) to a final concentration of about 0.83%, to facilitate pelleting of cellular fiberous material (optional step).
5. Spin sample in centrifuge to pellet insoluble material and flocculent (as appropriate).
6. Remove supernatant (i.e., lysate) and place in new tube or well.

DNA present in the lysate can be purified, for example, using a column based DNA purification kit (e.g., a QIAamp DNA mini kit).

### Example 2

### Direct Liquefaction Protocol on Corn Leaf Disks

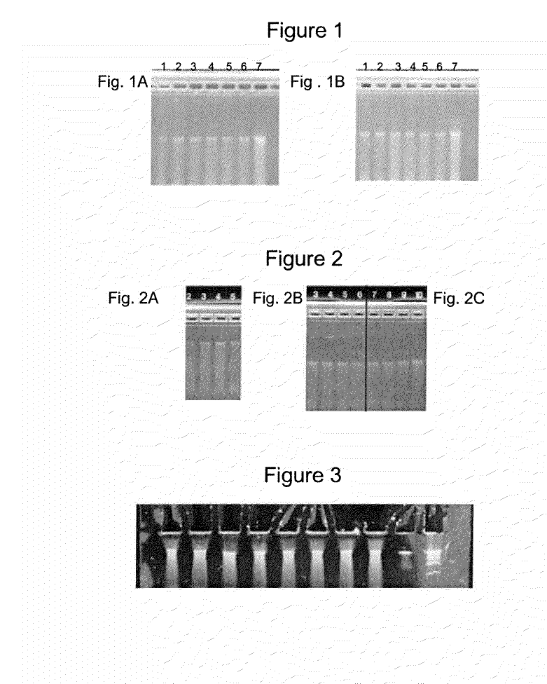

The protocol of Example 1 was performed on 6 mm corn leaf disks using enzymatic solutions comprising MES, calcium chloride, sorbitol, mannitol, boric acid, Tris, EDTA, and sarkosyl. The enzymatic solutions further comprised either cellulase, hemicellulase, and pectinase activities, or cellulase and pectinase activities. The enzymatic digestion was performed overnight at 25° C., without agitation. Genomic DNA from the resulting lysates was recovered using the QIAamp DNA mini kit from Qiagen, and the DNA was run out on an 0.8% agarose gel containing ethidium bromide. The results are shown in FIG. 1. FIG. 1A shows the results for the enzymatic solution comprising cellulase, hemicellulase, and pectinase activities, while FIG. 1B shows the results for the enzymatic solution comprising cellulase and pectinase. The individual lanes in each gel contain DNA from 3 leaf disks, 6 mm each (lanes 1-6) or 10 leaf disks, 6 mm each (lane 7).

### Example 3

### Direct Liquefaction Protocol on Corn Leaf Tears

The protocol of Example 1 was performed on corn leaf tears (˜1.5 cm²), the enzymatic digestion performed for 2 days at 25″C, without agitation. Three different enzymatic solutions were tested: (A) a solution comprising MES, calcium chloride, sorbitol, mannitol, boric acid, Tris, EDTA, and sarkosyl; (B) a solution comprising mannitol, boric acid, Tris, EDTA, and sarkosyl; and (C) a solution comprising boric acid, Tris, EDTA, and sarkosyl. Each of the three solution comprised cellulase, hemicellulase, and pectinase activities. Genomic DNA from the resulting lysates was recovered using the QIAamp DNA mini kit from Qiagen, and the DNA was run out on an 0.8% agarose gel containing ethidium bromide. The results are shown in FIG. 2. FIG. 2A shows the results for four samples prepared using enzymatic solution (A); FIG. 2B shows the results for four samples prepared using enzymatic solution (B); FIG. 2C shows the results for four samples prepared using enzymatic solution (C).

### Example 4

### Indirect Liquefaction Protocol

Another method of the invention for providing a cellular lysate from a cell wall-containing cellular sample is as follows:
1. Incubate leaf tear in a stabilizing solution of the invention (optional)
2. Decant stabilizing solution (if used)
3. Add 400 microliters of pre-enzymatic solution and incubate 2-3 hours at 25° C.
4. Add 560 microliters of stop solution
5. Aspirate or decant liquid (i.e., pre-enzymatic solution plus stop solution)
6. Add 1 ml of water, then aspirate or decant
5. Add 400 microliters of enzymatic solution and incubate overnight at 25° C.
6. Gently mix solution with finger or vortex to disintegrate cellular sample
7. Add 20 microliters of extraction solution and mix
8. Centrifuge to remove insoluble material
9. Remove lysate

DNA present in the lysate can be purified, for example, using a column based DNA purification kit (e.g., a QIAamp DNA mini kit).

### Example 5

### Indirect Liquefaction Protocol on Corn Leaf Tears

The protocol of Example 4 was performed on corn leaf tears (˜1.5, with (1) an alcohol/glycerol stabilization solution, (2) a pre-enzymatic solution comprising 1M hydroxide ion, (3) a stop solution comprising citric acid, (4) a buffer comprising MES, EDTA, citric acid, and cellulase and pectinase activities, and (5) a 1% Triton X-100 extraction solution. Genomic DNA from the resulting lysates was recovered using the QIAamp DNA mini kit from Qiagen, and the DNA was run out on a denaturing polyacrylamide gel and then stained with SYBR® gold. The results for eight samples run in parallel are shown in FIG. 3.

Although the invention has been described with reference to the presently preferred embodiments, it should be understood that various changes and modifications, as would be obvious to one skilled in the art, can be made without departing from the spirit of the invention. Accordingly, the invention is limited only by the following claims.

What is claimed:

1. A method for providing a plant tissue lysate, said method comprising:
incubating a plant tissue sample in an enzymatic solution,
wherein said enzymatic solution comprises at least two cell wall and tissue lattice degrading enzyme activities and a metal chelator; and
wherein said enzymatic incubation is carried out in the absence of mechanical processing.

2. The method of claim 1, wherein said enzymatic solution comprises a cellulase activity and a pectinase activity.

3. The method of claim 2, wherein said enzymatic solution further comprises a hemicellulase activity.

4. The method of claim 2, wherein said enzymatic solution further comprises a hemicellulase activity and a ligninase activity.

5. The method of claim 1, wherein said enzymatic solution comprises a metal chelator selected from the group consisting of EDTA, EGTA, o-phenanthroline, and crown ethers.

6. The method of claim 1, wherein said enzymatic solution comprises a preservative selected from the group consisting of boric acid, borate, phosphoric acid, phosphate, vanadate, and an alum.

7. The method of claim 1, wherein said enzymatic solution comprises a detergent.

8. The method of claim 7, wherein said detergent is an ionic detergent.

9. The method of claim 1, wherein said enzymatic solution has a pH of about 4.0 to about 9.0.

10. The method of claim 1, wherein said enzymatic solution comprises a buffer selected from the group consisting of 2-(cyclohexylamino) ethanesulfonic acid (CHES), N-(2-hydroxyethyl)piperazine-N′-(3-propanesulfonic acid) (EPPS), N-(2-hydroxyethyl)piperazine-N′-(2-ethanesulfonic acid (HEPES), 2-(N-morpholino) ethanesulfonic acid (MES), 3-(N-morpholino) propanesulfonic acid (MOPS), piperazine-N,N′-bis(2-ethanesulfonic acid (PIPES), [(2-hydroxy-1,1-bis[bydroxymethyl]ethyl) amino]-1-propanesulfonic acid (TAPS), ethanolamine, 3-amino-1-propanesulfonic acid, and 2-amino-2-hydroxymethyl-1,3-propanediol (Tris).

11. The method of claim 1, wherein said enzymatic solution comprises a short-chain polyol.

12. The method of claim 1, wherein said plant tissue sample is incubated with said enzymatic solution in a volume to volume ratio of about 1:2 to about 1:1000.

13. The method of claim 1, wherein said enzymatic incubation is carried out at ambient temperature.

14. The method of claim 1, wherein said enzymatic incubation is carried out at about 10° C. to about 40° C.

15. The method of claim 1, wherein said enzymatic incubation is carried out at about 20° C. to about 30″C.

16. The method of claim 1, wherein said plant tissue sample is incubated in said enzymatic solution for at least 12 hours.

17. The method of claim 1, further comprising agitating said plant tissue sample and said enzymatic solution at the end of said enzymatic incubation.

18. The method of claim 1, further comprising adding a flocculator to said enzymatic solution at the end of the enzymatic incubation.

19. The method of claim 1, wherein the plant tissue sample is fresh or freeze-dried.

20. The method of claim 1, further comprising:
incubating said plant tissue sample in a pre-enzymatic solution having a pH greater than about 10; and
stopping said pre-enzymatic incubation,
wherein said pre-enzymatic incubation is carried out prior to said enzymatic incubation.

21. The method of claim 20, wherein said pre-enzymatic solution has a hydroxide ion concentration of about 0.02M to about 5M.

22. The method of claim 20, wherein said pre-enzymatic solution comprises a base selected from the group consisting of a hydroxide salt, an amide salt, a carbanion salt, and a hydride salt.

23. The method of claim 20, wherein said pre-enzymatic incubation is carried out for about 1 hour to about 4 hours.

24. The method of claim 1, further comprising incubating said plant tissue sample in a stabilization solution prior to said enzymatic incubation, wherein said stabilization solution comprises an alcohol, a short-chain polyol, or a combination thereof.

25. The method of claim 24, wherein said stabilization solution comprises at least 50% alcohol, polyol, or a combination thereof.

26. The method of claim 24, wherein said plant tissue is incubated in said stabilization solution for at least 8 hrs.

27. The method of claim 1, wherein said lysate comprises nucleic acid, and wherein said nucleic acid is suitable for genetic analysis.

28. A method for providing a plant tissue lysate, said method comprising:
incubating a plant tissue sample in an enzymatic solution,
wherein said enzymatic solution comprises at least two cell wall and tissue lattice degrading enzyme activities and a metal chelator; and
wherein said enzymatic incubation is carried out at ambient temperature.

29. A kit comprising:
a first solution comprising a metal chelator, a preservative, and an ionic detergent, wherein said first solution has a pH of about 4.0 to about 9.0; and
at least two cell wall and tissue lattice degrading enzyme activities.

30. The kit of claim 29, wherein said wherein said at least two activities include a cellulase activity and a pectinase activity.

31. The kit of claim 29, wherein said first solution further comprises a short-chain polyol.

32. The kit of claim 29, further comprising an instruction for using said first solution and said at least two cell wall and tissue lattice degrading enzyme activities to prepare a plant tissue lysate.

33. A mixture comprising:
plant tissue; and
an enzymatic solution comprising a metal chelator, a preservative, an ionic detergent, and at least two cell wall and tissue lattice degrading enzyme activities,
wherein the pH of said mixture is about 4.0 to about 9.0.

34. The mixture of claim 33, wherein said at least two cell wall and tissue lattice degrading enzyme activities include a cellulase activity and a pectinase activity.

35. The mixture of claim 34, wherein said at least two cell wall and tissue lattice degrading enzyme activities further include hemicellulase activity and, optionally, ligninase activity.

36. The mixture of claim 33, wherein said enzymatic solution further comprises a short-chain polyol.

37. The mixture of claim 33, wherein said mixture is at ambient temperature.

38. The mixture of claim 33, wherein said mixture is maintained without mechanical agitation.
